(19) United States
(12) Patent Application Publication
(10) Pub. No.: US 2007/0083147 A1
(43) Pub. Date: Apr. 12, 2007

(54) Iontophoresis apparatus and method to deliver antibiotics to biological interfaces

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Tokyo (JP)

(21) Appl. No.: 11/542,664

(22) Filed: Oct. 2, 2006

Related U.S. Application Data
(60) Provisional application No. 60/722,224, filed on Sep. 30, 2005.

(51) Int. Cl. A61N 1/30

(52) U.S. Cl. 604/20

(57) ABSTRACT

An iontophoresis device operable to deliver active agent to a biological interface such as skin or mucous membranes includes the combination of a hydrogel-based wound covering with an iontophoresis device to deliver antibiotic to biological interfaces. The effective concentration of antibiotics from oral or intravenous administration rarely reaches poorly perfused tissues such as cartilage or skin ulcerations resulting in entrenched and difficult to treat infections. Local deliver of additional antibiotic would serve to maintain the effective concentration of drug to the target tissues. Combining an iontophoresis device with a hydrogel-based wound covering to deliver localized antibiotic allows faster wound healing and prevent infection.

### CROSS-REFERENCE TO RELATED APPLICATION

This application claims the benefit under 35 U.S.C. §119(e) of U.S. Provisional Patent Application No. 60/722,224 filed Sep. 30, 2005, where this provisional application is incorporated herein by reference in its entirety.

### BACKGROUND OF THE INVENTION

1. Field of the Invention

The present disclosure generally relates to the field of iontophoresis and, more particularly, to the delivery of antibiotics and other therapeutic agents or drugs to a biological interface.

2. Description of the Related Art

Iontophoresis employs an electromotive force and/or current to transfer an active agent (e.g., a charged substance, an ionized compound, an ionic drug, a therapeutic, a bioactive agent, and the like), to a biological interface (e.g., skin, mucous membrane, and the like), by using a small electrical potential to an electrode proximate an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See, e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their ability to deliver drugs across various biological interfaces including, for example, tissue barriers.

The present disclosure is directed to overcoming one or more of the shortcomings set forth above, and to providing further related advantages.

### BRIEF SUMMARY OF THE INVENTION

In at least one embodiment, an iontophoresis device operable to deliver active agent to a biological interface such as skin or mucous membranes includes the combination of a hydrogel-based wound covering with an iontophoresis device to deliver antibiotic to biological interfaces. The effective concentration of antibiotics from oral or intravenous administration rarely reaches poorly perfused tissues such as cartilage or skin ulcerations resulting in entrenched and difficult to treat infections. Local deliver of additional antibiotic would serve to maintain the effective concentration of drug to the target tissues. Combining an iontophoretic device with a hydrogel-based wound covering to deliver localized antibiotic allows faster wound healing and prevent infection.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements and have been solely selected for ease of recognition in the drawings.

FIG. 1A is a top, front view of a transdermal drug delivery system according to one illustrated embodiment.

FIG. 1B is a top, plan view of a transdermal drug delivery system according to one illustrated embodiment.

FIG. 2A is a schematic diagram of the iontophoresis device of FIGS. 1A and 1B comprising active and counter electrode assemblies, according to one illustrated embodiment.

FIG. 2B is a schematic diagram of the iontophoresis device of FIG. 2A positioned on a biological interface, with an optional outer release line removed to expose the active agent, according to another illustrated embodiment.

FIG. 3 is a schematic diagram of the iontophoresis device of FIG. 2A further comprising a permeable bacterial barrier layer, according to one illustrated embodiment.

### DETAILED DESCRIPTION OF THE INVENTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices, including but not limited to voltage and/or current regulators, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

It should be noted that, as used in this specification and the appended claims, the singular forms “a,” “an,” and “the” include plural referents unless the content clearly dictates otherwise. Thus, for example, reference to an iontophoresis device including “an electron element” includes a single electrode element, or two or more electrode elements. It should also be noted that the term “or” is generally employed in its sense including “and/or” unless the content clearly dictates otherwise.

As used herein and in the claims, the term “membrane” means a boundary, a layer, a barrier or material, which may or may not be permeable. The term “membrane” may further refer to an interface. Unless specified otherwise, membranes may take the form of a solid, liquid, or gel, and may or may not have a distinct lattice, non-cross-linked structure, or cross-linked structure.

As used herein and in the claims, the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion, opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non-cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional networks of, for example, cross-linked hydrophilic polymers in the form of a gel and substantially composed of water. Hydrogels may have a net positive or negative charge, or may be neutral.

As used herein and in the claims, the term “reservoir” means any form or mechanism to retain an element, compound, pharmaceutical composition, diagnostic composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and or current into the biological interface. A reservoir may also retain an electrolyte solution.

As used herein and in the claims, “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like), non-pharmaceuticals (e.g., a cosmetic substance, and the like), diagnostic agents, an antibiotic, a vaccine, an immunological agent, a local or general anesthetic or painkiller, an antigen or a protein or a peptide, such as insulin, a chemotherapy agent, or an anti-tumor agent.

In some embodiments, the term “active agent” refers to the active agent itself, as well as its pharmacologically active salts, pharmaceutically or diagnostically acceptable salts, pro-drugs, metabolites, analogs, and the like. In some further embodiments, the active agent includes at least one ionic, cationic, ionizable, and/or neutral therapeutic drug and/or pharmaceutically acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. For instance, an active agent having an amino group can typically take the form of an ammonium salt in solid state and dissociate into a free ammonium ion (NH₄⁺) in an aqueous medium of appropriate pH. Other active agents may have functional groups that are readily convertible to a negative ion or can dissociate into a negatively charged ion and a counter ion in an aqueous medium. Yet other active agents may be polarized or polarizable, that is, exhibiting a polarity at one portion relative to another portion.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

As used herein and in the claims, an “antibiotic” is an active agent that may be used to treat infections caused by bacteria or other microorganisms. In certain cases, an antibiotic is a substance produced by a microorganism, such as a mold or a bacteria, to selectively inhibit the growth of another organism. As used herein and in the claims, the terms “antibiotic,” “anti-infective,” and “anti-bacterial” may be used interchangeably. Non-limiting examples of antibiotics include aclacinomycin A, actinomycin, aminoglycocides, amphotericin, anthracyclines, anthramycin, antimycin, aztreonam, Azactam, bacitracin, camptothecins, topotecan, carbomycin, carubicin, cephaloglycin, Kafocin, cephaloridine, cephalosporins, Mefoxin, chloramphenicol, Choromycetin, chlortetracycline, Aureomycin, chromomycin A₃, ciprofloxacin, Cipro, cycloserine, daunorubicin, dihydrostreptomycin, doxifluridine, doxorubicin, doxocycine, Vibramycin, epirubicin, erythromycin, E-Mycin, Erythrocin, Ethril, Ilosone, Pediamycin, fluoropyrimidines, 5-fluorouracil, 5-fluorodeoxyuridine, fluroquinolones, folic acid antagonists, gentamycin, Garamycin, gramicidin, hydroxyureas, idarubicin, kanamycin, Kantrex, lincomycin, Lincocin, macrolides, menogaril, methotrexate, and derivatives or analogs thereof, mitomycin, Mutamycin, mitoxantrone, mycomycin, fradicin, Neobiotic, neomycin, nogalamycin, novobiocin, nystatin, Nystan, Mycostatin, olivomycin A, pirarubicin, plicamycin, podophyllotoxins, etoposide, teniposide, tetracyclines, Achromycin, Sumycin, hydroxytetracycline, oxytetracycline, Terramycin, penicillins, polymyxin, pyocyanase, zorubicin

Further non-limiting examples of active agents include lidocaine, articaine, and others of the—caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate, and other 5-hydroxytryptamine1 receptor subtype agonists; resiquimod, imiquimod, and similar TLR 7 and TLR 8 agonist and antagonists; domperidone, granisetron hydrochloride, ondansetron, and other such anti-emetic drugs; zolpidem tartrate and similar sleep inducing agents; L-DOPA and other anti-Parkinson's medications; aripiprazole, olanzapine, quetiapine, risperidone, clozapine, and ziprasidone, as well as other neuroleptica; diabetes drugs, such as exenatide; as well as peptides and proteins for treatment of obesity and other maladies.

Additional non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, “antigen” or “antigenic” or “antigenicity” refers to a protein, polypeptide or carbohydrate, and the like, that is recognized by the body as foreign and that stimulates the immune system to produce an antibody; as used herein and in the claims, “antigenic determinant”, also commonly referred to as “epitope,” refers to a specific area or structure (that is, an “antigenic site”) on the surface of an antigen that can cause an immune response, thus stimulating production of an antibody that can recognize and bind to the antigenic site or to structurally related antigenic sites. As used herein and in the claims, an “antigenic portion” of an antigen is a portion that is capable of reacting with serum obtained from an individual infected with an organism from which the antigen is derived or with the antigen itself.

As used herein and in the claims, a polypeptide comprising an antigenic determinant that is “similar to” an antigenic determinant located on a specified antigen refers to a polypeptide that elicits an immune response comparable to that elicited by the specified antigen.

As used herein and in the claims, the term “immunogen” or “immunogenicity” refers to any agent that elicits an immune response. Examples of an immunogen include, but are not limited to natural or synthetic (including modified) peptides, proteins, carbohydrates, lipids, oligonucleotides (RNA, DNA, etc.), chemicals, or other agents.

As used herein and in the claims, the term “polypeptide” encompasses amino acid chains of any length, including full-length proteins, wherein the amino acid residues are linked by covalent peptide bonds.

As used herein and in the claims, a “variant” is a polypeptide that differs from a native antigen only in conservative substitutions and/or modifications, such that antigenic properties of the native antigen are retained. Such variants may generally be identified by modifying a polypeptide sequence and evaluating the antigenic properties of the modified polypeptide. A “conservative substitution” is one in which an amino acid is substituted for another amino acid that has similar properties. In general, the following groups of amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Variants may also, or alternatively, be modified by, for example, the deletion or addition of amino acids that have minimal influence on the antigenic properties or structural characteristics of the polypeptide.

As used herein and in the claims, a “fusion protein” or “fusion polypeptide” comprises two or more protein/polypeptide sequences joined via a peptide linkage into a single amino acid chain. The sequences may be joined directly, without intervening amino acids, or by way of a linker amino acid sequence.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivations thereof, refers to an agent that modifies the effect of another agent while having few, if any, direct effects when given by itself. For example, an adjuvant may increase the potency or efficacy of a pharmaceutical, or an adjuvant may alter or affect an immune response.

As used herein and in the claims, the term “agonist” refers to a compound that can combine with a receptor (e.g., a Toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the terms “vehicle,” “carrier,” “pharmaceutical vehicle,” “pharmaceutical carrier,” “pharmaceutically acceptable vehicle,” “pharmaceutically acceptable carrier,” “diagnostic vehicle,” “diagnostic carrier,” “diagnostically acceptable vehicle,” or “diagnostically acceptable carrier” may be used interchangeably, depending on whether the use is pharmaceutical or diagnostic, and refer to pharmaceutically or diagnostically acceptable solid or liquid, diluting or encapsulating, filling or carrying agents, which are usually employed in pharmaceutical or diagnostic industry for making pharmaceutical or diagnostic compositions. Examples of vehicles include any liquid, gel, salve, cream, solvent, diluent, fluid ointment base, vesicle, liposomes, niosomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, a pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacologically active agent in the formulation.

As used herein and in the claims, the term “cyclodextrin” refers to any of a family of cyclic oligosaccharides. Cyclodextrins, also sometimes called cycloamyloses, are composed of, but are not necessarily limited to, five or more D-glucopyranoside units, connected by α-(1,4) glycosidic linkages, as in amylase. Cyclodextrins having as many as 32 1,4-glucopyranoside units have been well characterized. Typically, cyclodextrins contain, but are not necessarily limited to, six to eight glucopyranoside units in a ring, commonly termed c-cyclodextrin (six units), β-cyclodextrin (seven units), and γ-cyclodextrin (eight units). These may be naturally occurring or produced synthetically.

As used herein and in the claims, “in conjunction with” and any derivations thereof refers to administration of an active agent, vehicle, carrier, and the like, simultaneously with, prior to, or subsequent to administration of a further active agent, vehicle, carrier, and the like.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

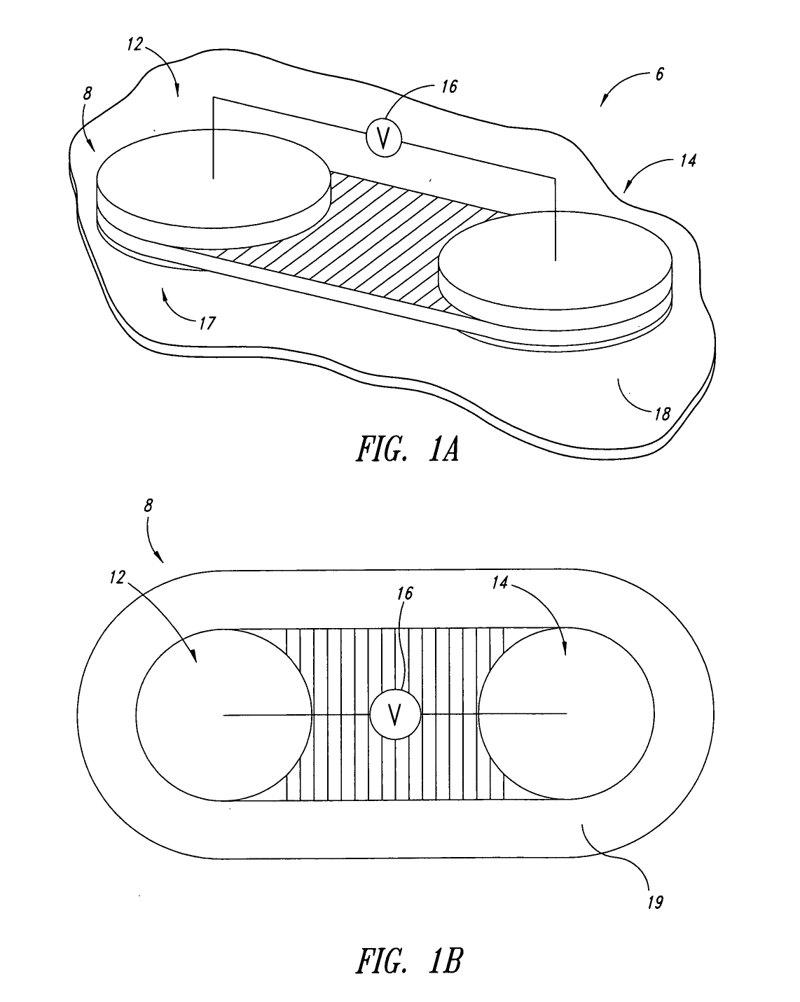

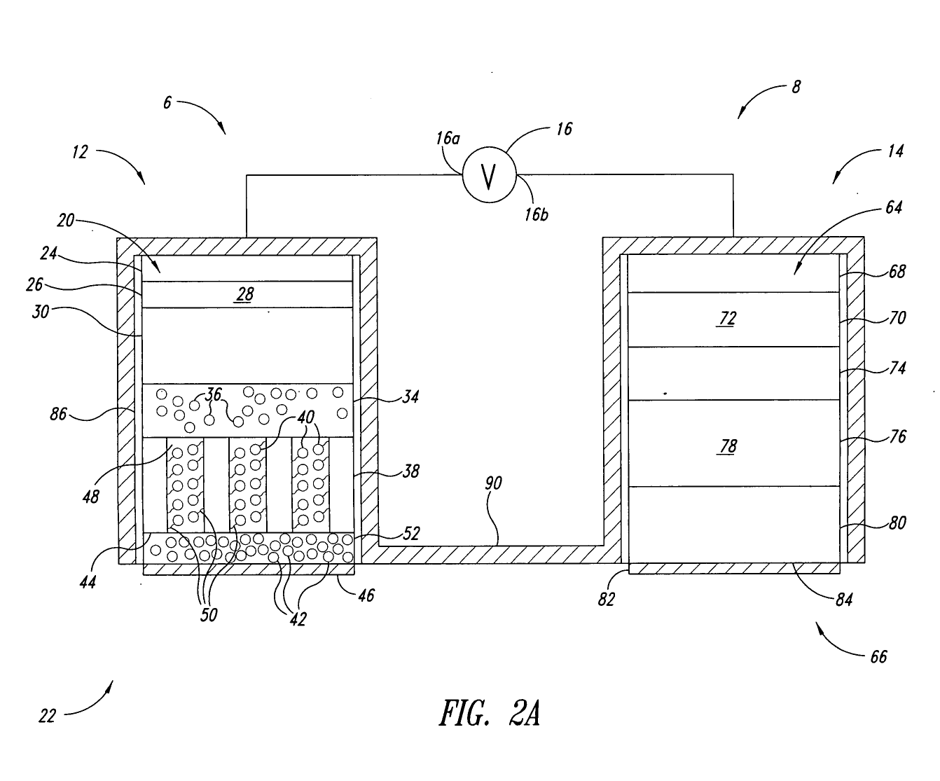

FIGS. 1A and 1B show an exemplary transdermal drug delivery system 6 for delivering of one or more active agents to a subject. The system 6 includes an iontophoresis device 8 including active and counter electrode assemblies 12, 14, respectively, and a power source 16. The active and counter electrode assemblies 12, 14, are electrically coupled to the power source 16 to supply an active agent contained in the active electrode assembly 12, via iontophoresis, to a biological interface 18 (e.g., a portion of skin or mucous membrane). In some embodiments, the iontophoresis device 8 may optionally include an outer adhesive surface 19 for physically coupling the iontophoresis device 8 to the biological interface 18 of the subject.

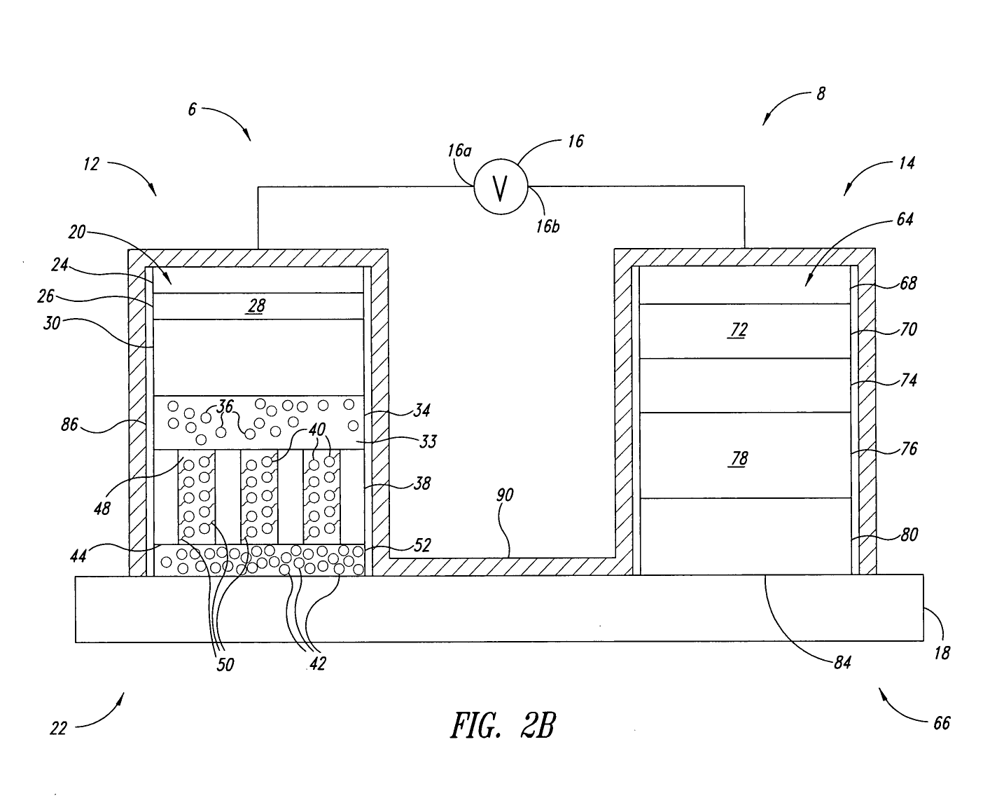

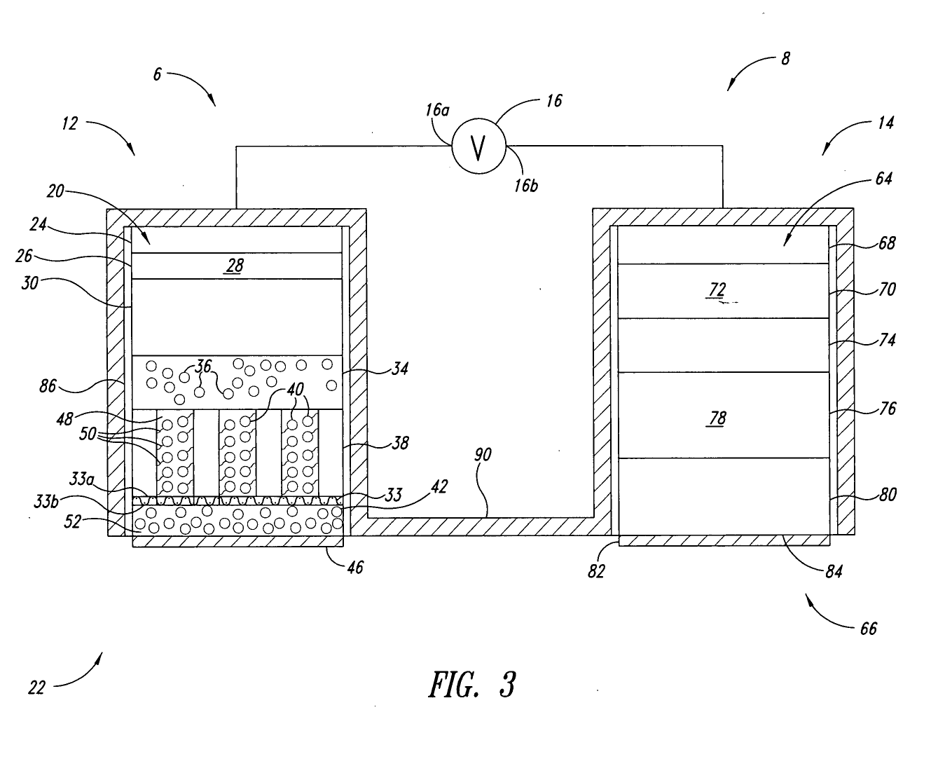

As shown in FIGS. 2A, 2B, and 3, the active electrode assembly 12 comprises, from an interior 20 to an exterior 22 of the active electrode assembly 12: an active electrode element 24, an electrolyte reservoir 26 storing an electrolyte 28, an inner ion selective membrane 30, an inner active agent reservoir 34 storing active agent 36, an optional outermost ion selective membrane 38 that optionally caches additional active agent 40, an optional further active agent 42 carried by an outer surface 44 of the outermost ion selective membrane 38, and an optional outer release liner 46.

The active electrode assembly 12 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological interface 18. Each of the above elements or structures will be discussed in detail below.

The active electrode element 24 is electrically coupled to a first pole 16a of the power source 16 and positioned in the active electrode assembly 12 to apply an electromotive force to transport the active agent 36, 40, 42 via various other components of the active electrode assembly 12. Under ordinary use conditions, the magnitude of the applied electromotive force is generally that required to deliver the one or more active agents according to a therapeutic or diagnostic effective dosage protocol. In some embodiments, the magnitude is selected such that it meets or may exceed the ordinary use operating electrochemical potential of the iontophoresis delivery device 8.

The active electrode element 24 may take a variety of forms. In one embodiment, the active electrode element 24 may advantageously take the form of a carbon-based active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such an anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode, and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 28 may be in the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate:0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na+) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiments illustrated in FIGS. 2A, 2B, and 3, take the form of an ion exchange membrane having pores 48 (only one called out in FIGS. 2A, 2B and 3 for sake of clarity of illustration) of the ion selective membrane 38 including ion exchange material or groups 50 (only three called out in FIGS. 2A, 2B, and 3 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin. Alternatively, where the active agent 36, 40, 42 is an anion, the outermost ion selective membrane 38 may take the form of an anion exchange membrane, thus allowing the passage of anionic active agent.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of a semi-permeable or microporous membrane that is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with the additional active agent 40, such as ionized or ionizable drugs or therapeutic or diagnostic agents and/or polarized or polarizable drugs or therapeutic or diagnostic agents. Where the outermost ion selective membrane 38 is an ion exchange membrane, a substantial amount of active agent 40 may bond to ion exchange groups 50 in the pores, cavities, or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiments, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives.

An interface-coupling medium (not shown) may be employed between the electrode assembly and the biological interface 18. The interface-coupling medium may, for example, take the form of an adhesive and/or gel. The gel may, for example, take the form of a hydrating gel. Selection of suitable bioadhesive gels is within the knowledge of one skilled in the relevant art.

In the embodiment illustrated in FIGS. 2A, 2B, and 3, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupled to a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxyl (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes are discussed above.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. The outer release liner 82 is shown in place in FIGS. 2A and 3, and removed in FIG. 2B. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The iontophoresis device 8 may further comprise an inert molding material 186 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2B, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14. It will be apparent that the active and counter electrode assemblies 12, 14 of the embodiment exemplified in FIG. 3 may readily be positioned on a biological interface in the manner depicted in FIG. 2B.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may, for example, take the form of an adhesive and/or gel. The gel may, for example, take the form of a hydrating gel or a hydrogel. If used, the interface-coupling medium should be permeable by the active agent 36, 40, 42.

In certain embodiments, as exemplified in FIG. 3, an iontophoresis device according to the present disclosure for use as a wound dressing may further include a permeable bacterial barrier layer 33 having first and second sides 33a and 33b. In certain such embodiments, layer 52 may advantageously comprise a hydrogel. As shown, the second side 33b of the permeable bacterial barrier layer 33 may be adhered to the hydrogel layer 52.

In certain aspects, the permeable bacterial barrier layer 33 may be formed of a material having porosity sufficient to allow the barrier layer to readily adhere to hydrogel layer 52 without an adhesive, thereby reducing the cost of manufacture of the wound dressing. Alternatively, in certain embodiments, an adhesive layer (not shown) may be included to bond the hydrogel layer 52 to the bacterial barrier layer 33.

In some embodiments, the permeable bacterial barrier layer 33 may be formed of a porous material comprising a foam material including silica and a polyolefin, where the porous material may have a porosity ranging from about 30% to about 90%. In at least one embodiment, the porous material is a microporous synthetic sheet commercially available from PPG Industries, Inc. under the trademark Teslin®.

In certain aspects, a hydrogel material forming layer 52 may comprise a saline solution in an aqueous gel-like phase. In certain embodiments, hydrogel materials as disclosed elsewhere herein or as are known in the art may comprise the hydrogel layer. In at least one exemplary embodiment, a hydrogel material may correspond to that disclosed in U.S. Pat. No. 5,423,737, issued Jun. 13, 1995, the disclosure of which is incorporated herein by reference in its entirety. The gel-like consistency of a hydrogel material may allow bonding to the site of the wound without creating an actual adhesive attachment that may damage new cell tissue upon removal. One advantage of the hydrogel layer is that it will not deteriorate as the wound fluids are absorbed. Additionally, a hydrogel layer may permit clean and neat removal of the wound dressing when the wound heals or the dressing is changed, without causing further damage to the site of the wound. An additional advantage of the hydrogel layer is that it may be substantially transparent, thus allowing inspection of the wound without removing the wound dressing.

The chemistry of hydrogels is known in the art. Polymers are long, chain molecules made of regular repeating units/patterns of building blocks (monomers). Naturally occurring polymers are common and have been included in materials used as wound treatments (e.g., various forms of collagen). Many industrial polymers use a single monomer or combine two monomers into A-A-A or A-B-A structures, respectively, for example. Synthetic hydrogels used in wound dressings may commonly be made from polyvinyl pyrrolidone, polyacrylamide, or polyethylene oxide. For example, the structure of polyethylene oxide, which is contained in Vigilon® (CR Bard, Covington, Ga.), is as follows:
(—CH2-CH2-O—CH2-CH2-O—CH2-CH2-O—)
Noncovalent interactions between adjacent polymer molecules enable such strands to associate with one other, particularly if the monomers contain aromatic rings. This effect may provide strength to devices constructed from such polymers. In order to impart further structural integrity to the polymer, polymer molecules are covalently cross-linked using free radical reactions to activate side chains that protrude from the monomers. While this cross-linking can be accomplished chemically, the least expensive and most uniform result is achieved by irradiating the non-crosslinked polymer with ultraviolet light or electron beam.

Hydrogels are polymers with hydrophilic side chains that may bind up to three times their weight in water. Thus, hydrogels essentially comprise a three-dimensional network with water or electrolyte incorporated in the interstices. This important feature provides the special advantages of hydrogels when compared with other dressings: fluid absorption, hydration of the wound bed, cooling of the wound surface, and pain control.

The high water or electrolyte content of hydrogels also facilitates electrical conduction. Depending upon the extent of crosslinking and the degree of hydration, hydrogels can be created in physical forms ranging from amorphous gels that may conform to the irregular surfaces of a wound bed to semi-stiff sheets that have enough structural integrity to function alone without a secondary dressing.

In particular embodiments, hydrogels may be loaded with therapeutic agents for topical delivery to the wound site. Hydrogels are able to hold and protect a wide variety of chemical agents, including antibiotics. Combining a hydrogel wound covering with an iontophoresis device provides a delivery mechanism for localized delivery of the antibiotic, thus providing desirable therapeutic effects within wounds.

In some embodiments of the devices and methods according to the present disclosure, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively electrically coupled to the active and counter electrode assemblies 12, 14 via a control circuit, for example, via carbon fiber ribbons. The iontophoresis device 8 may include discrete and/or integrated circuit elements to control the voltage, current and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more antibiotics, cationic or anionic drugs, or other therapeutic or diagnostic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides or nucleic acids. In other embodiments, the active agent may be mixed with another agent to form a complex capable of being transported across the biological interface via one of the motive methods described above.

In some embodiments, the transdermal delivery system 6 includes an iontophoretic delivery device 8 for providing transdermal delivery of one or more antibiotic, therapeutic, or diagnostic active agents 36, 40, 42 to a biological interface 18. The delivery device 8 includes active electrode assembly 12 including at least one active agent reservoir and at least one active electrode element operable to provide an electromotive force to drive an active agent from the at least one active agent reservoir. The delivery device 8 may include a counter electrode assembly 14 including at least one counter electrode element 68, and a power source 16 electrically coupled to the at least one active and the at least one counter electrode elements 24, 68. In some embodiments, the iontophoretic delivery device 8 may further include one or more active agents 36, 40, 42 loaded in the at least one active agent reservoir 34.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the invention, as will be recognized by those skilled in the relevant art. The teachings provided herein can be applied to other agent delivery systems and devices, not necessarily the exemplary iontophoresis active agent system and devices generally described above. For instance, some embodiments may omit one or more reservoirs, membranes or other structure. In other instances, some embodiments may include additional structure. For example, some embodiments may include a control circuit or subsystem to control a voltage, current, or power applied to the active and counter electrode elements 24, 68. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd. In certain embodiments, preparations of these various hydrogels may be made to incorporate proteins or polypeptides, or fusion proteins or fusion polypeptides, for use with the devices and methods disclosed herein. In certain embodiments, such hydrogel preparations may serve as reservoirs for the various active agents. Such hydrogel preparations may constitute, for example, inner active agent reservoir 34 or layer 52 of the active electrode assembly in FIGS. 2A, 2B, and 3.

Various embodiments discussed herein may advantageously employ microstructures, for example, microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids. Microneedle devices may be used, for example, to deliver any of a variety of compounds and/or compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a solution of an active agent, such as a drug or therapeutic or diagnostic agent, that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No. WO03037425; U.S. patent application Ser. No. 10/488970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

As one skilled in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An iontophoretic device, comprising:
an antibiotic, or a composition thereof, useful to prevent infection in a biological tissue; and
an active electrode element selectively operable to drive the one or more antibiotics, or composition thereof, from the iontophoretic device.

2. The device of claim 1, further comprising:
a layer comprising the antibiotic, or composition thereof, the layer positioned so as to be directly adjacent to a biological interface when the device is placed on the biological interface.

3. The iontophoretic device of claim 2 wherein the layer comprising the antibiotic, or composition thereof, comprises a hydrogel.

4. The device of claim 2, further comprising:
an inner active agent reservoir positioned between the active electrode element and the layer comprising the antibiotic, or composition thereof; and
an additional active agent, or composition thereof, stored in the inner active agent reservoir, the additional active agent having a polarity that is the same as the polarity of the antibiotic in the layer comprising the antibiotic, or composition thereof.

5. The device of claim 4 wherein the additional active agent, or composition thereof, stored in the inner active agent reservoir is substantially the same as the antibiotic, or composition thereof, in the layer comprising the antibiotic, or composition thereof.

6. The device of claim 4 wherein the additional active agent, or composition thereof, stored in the inner active agent reservoir comprises an antibiotic, or a composition thereof, that is not substantially the same as the antibiotic, or composition thereof, in the layer comprising the antibiotic, or composition thereof.

7. The device of claim 4 wherein the additional active agent, or composition thereof, does not comprise an antibiotic, or a composition thereof.

8. The device of claim 2, further comprising:
a permeable barrier layer positioned between the active electrode and the layer comprising the antibiotic or composition thereof.

9. The device of claim 8 wherein the permeable barrier layer is a permeable bacterial barrier layer.

10. The device of claim 2, further comprising:
an electrolyte positioned between the active electrode element and the layer comprising the antibiotic, or composition thereof.

11. The device of claim 1, further comprising:
a hydrogel layer positioned such that it is between the active electrode element and a biological interface when the device is placed on the biological interface for use.

12. The device of claim 11, further comprising:
an electrolyte reservoir positioned between the active electrode element and the hydrogel layer.

13. The device of claim 12, further comprising:
an inner active agent reservoir positioned between the electrolyte reservoir and the hydrogel layer; and
an active agent stored within the inner active agent reservoir, the active agent having a polarity the same as the polarity of the antibiotic.

14. The device of claim 13 wherein the active agent is an antibiotic.

15. The device of claim 13 wherein the active agent is not an antibiotic.

16. The device of claim 13, further comprising:
an outermost ion selective membrane positioned between the inner active agent reservoir and the hydrogel layer; and
an additional active agent cached within the outermost ion selective membrane, the additional active agent having a polarity the same as the polarity of the active agent stored within the inner active agent reservoir.

17. The device of claim 16 wherein the additional active agent is substantially the same as the active agent stored within the inner active agent reservoir.

18. The device of claim 16 wherein the additional active agent is an antibiotic.

19. The device of claim 16 wherein the additional active agent is not an antibiotic.

20. The device of claim 16, further comprising:
a permeable barrier layer positioned between the outermost ion selective membrane and the hydrogel layer.

21. The device of claim 20 wherein the permeable barrier layer is a permeable bacterial barrier layer.
